(19) United States
(12) Patent Application Publication
(10) Pub. No.: US 2007/0197585 A1
(43) Pub. Date: Aug. 23, 2007

(54) Use of cysteinyl leukotriene 2 receptor antagonists

(76) Inventors: Jilly Evans, Lansdale, PA (US); Anthony Ford-Hutchinson, Doylestown, PA (US); John H. Hutchinson, La Jolla, CA (US); Robert Young, Senneville (CA)

Correspondence Address: MERCK AND CO., INC, P O BOX 2000, RAHWAY, NJ, 07065-0907, US

(21) Appl. No.: 10/589,646

(22) PCT Filed: Feb. 24, 2005
(86) PCT No.: PCT/US05/05683

§ 371 Date: Aug. 16, 2006

Related U.S. Application Data
(60) Provisional application No. 60/547,876, filed on Feb. 26, 2004.
(60) Provisional application No. 60/633,113, filed on Dec. 3, 2004.

(51) Int. Cl. A61K 31/47; A61K 31/196

(52) U.S. Cl. 514/311; 514/485

(57) ABSTRACT

The instant invention provides a method for treating and/or reducing the risk for atherosclerosis, pulmonary fibrosis and stroke, comprising administering an effective amount of a cysteinyl leukotriene 2 receptor antagonist, including a selective cysteinyl leuoktriene 2 receptor antagonist and a dual cysteinyl leukotriene 1 receptor and cysteinyl leukotriene 2 receptor antagonist to a patient in need of such treatment.

### CROSS-REFERENCE TO RELATED APPLICATIONS

This application claims priority to provisional application Ser. No. 60/547,876, filed Feb. 26, 2004, and provisional application Ser. No. 60/633,113, filed Dec. 3, 2004.

### FIELD OF THE INVENTION

This invention is directed to the use of cysteinyl leukotriene 2 (CysLT₂) receptor antagonists for the treatment and prevention of various diseases, including atherosclerosis and related disease events, pulmonary fibrosis and stroke.

### BACKGROUND OF THE INVENTION

Leukotrienes are potent contractile and inflammatory mediators derived by enzymatic oxygenation of arachidonic acid by 5-lipoxygenase. LTC_{4,} LTD₄ and LTE₄ are collectively known as the cysteinyl leukotrienes (CysLT's). The CysLT's have been shown to have broncho-constrictive, cardiac and inflammatory actions that are mediated through the action of two G-protein coupled receptors CysLT₁ and CysLT₂ (Metters, J Lipid Mediators and Cell Signalling 12:413427 (1995)). CysLT₁ antagonists have been shown to be clinically efficacious in the treatment of chronic asthma.

The human CysLT₁ and CysLT₂ receptors have been cloned and characterized. (Heise et al, J Biol Chem, 275:30531-30536 (2000)). The CysLT₂ receptor is a 337 amino acid putative 7 transmembrane spanning protein with consensus amino acid sequences of the rhodopsin subfamily GPCRs. The CysLT₁ receptor is a 346 amino acid membrane protein with approximately 38% amino acid identity to the CysLT₁ receptor. Northern blot analysis of the CysLT₁ receptor mRNA showed its highest expression in spleen and peripheral blood leukocytes. The human CysLT₂ receptor is most highly expressed in the heart, placenta and adrenal medulla.

In situ and immunohistochemical analyses of the distribution of the CysLT₁ receptor shows expression in lung smooth muscle and interstitial macrophages and in peripheral blood eosinophils, subsets of monocytes and macrophages, subsets of pre B cells, in precursor CD34+ stem cells and in bone marrow derived mast cells (Figueroa et al, 2001, Am J Resp Crit Care 163(1):226-233; Mellor et al, 2001 Proc Nat Acad Sci USA 98:7964-7969 (2001)). The CysLT₁ receptor is localized to chromosome Xq13-21 while the CysLT₂ receptor is localized at 13q14, the latter being an atopic linkage region. Both the CysLT₁ receptor and the CysLT₂ receptor functionally activate cells through mobilization of intracellular calcium (Lynch et al, 1999 Nature 399:789-793; Sarau et al, 1999, Mol Pharmacol 56:657-663; Heise et al, 2000; Takasaki et al, 2000, Biochem Biophys Res Commun 274:316-322; Nothacker et al, 2000, Mol Pharmacol 58: 1601-1608).

Selective CysLT₁ receptor antagonists montelukast (Singulair™), zafirkulast (Accolate™) and pranlukast (Orion™), which block the activation of the recombinant human CysLT₁ receptor but not the CysLT₂ receptor, are approved for treatment of asthma. The compound BAY u9773 is a full antagonist of the CysLT₁ receptor function but a partial agonist of the CysLT₂ receptor function (Nothacker et al, 2000, Mol Pharmacol 58:1601-1608).

The human CysLT₂ receptor has been identified on human cardiac Purkinje fibers, myocytes, and coronary artery smooth muscle cells (Heise et al.; Takasaki, Biochem Biophys Res Commun 2001 274:316-322; Nothacker et al., 2000). The CysLT₂ receptor has also been identified on human endothelial cells. High expression of the CysLT₂ receptor mRNA has been demonstrated on the atherosclerotic smooth muscle arterial and endothelial cells (Löet al., Arteriosclerosis, Thrombosis and Vascular Biology 23:e32-e36 (2003)).

The CysLT₁ receptor could be present on the monocyte/macrophage foam cells, mast cells and smooth muscle cells. Production of cysteinyl leukotrienes by the macrophage foam cells may activate the CysLT₂ receptor on endothelial cells, resulting in greater adhesion of plaque activating factors and in endothelial cell migration, thereby enhancing the potential for plaque rupture.

In atherosclerosis cells, the lesional area expressing the CysLT₂ receptor may include monocyte/macrophage foam cells, mast cells, smooth muscle cells and endothelial cells, and thus the CysLT₂ receptor may be present in atherosclerosis cells.

Thus, activation of the CysLT₁ and CysLT₂ receptors on coronary smooth muscle cells may activate contraction and plaque rupture. Autocrine activation of the CysLT₁ and CysLT₂ receptors on the foam macrophage or interstitial mast cells may result in further release of damaging inflammatory and immune molecules. As a result, a CysLT₂ antagonist, including both a CysLT₂ receptor selective antagonist or dual CysLT₁/CysLT₂ receptor antagonist could prevent the endothelial cell, smooth muscle cell and inflammatory cell activation, thereby preventing plaque rupture.

Despite significant therapeutic advances in the treatment and prevention of atherosclerosis and ensuing atherosclerotic disease events, further treatment options are clearly needed. The instant invention addresses that need by providing methods for using cysteinyl leukotriene 2 receptor antagonists, including selective CysLT₂ receptor antagonists and dual CysLT₂ and CysLT₁ receptor antagonists for the treatment and prevention of atherosclerosis.

Recent studies have also suggested that CysLT's may have a role in the pathogenesis of pulmonary fibrosis. (Beller et al, 2004 Proc Nat'l Acad Sci, 101(9): 3047-3052). Cys LT's have been found in homogenates of lung biopsy specimens from patients with pulmonary fibrosis. (Wilborn et al, 1996, J Clin Invest 97(8):1827-1836). Further, it has been shown that disruption of 5-lipoxygenase production reduces bleomycin induced injury and reduces inflammatory cells detected in lung tissue in mice, in a recognized model of pulmonary fibrosis. Peters-Golden et al, 2002, Am J Resp Crit Care Med 165:229-235. Thus, Cys-LT₂ receptor antagonists may be useful in treating pulmonary fibrosis.

The CysLT's may also have a role in stroke, and the injuries and conditions resulting from the reduction in blood flow to the brain caused by stroke. One of the significant events in the biochemical cascade of stroke is the intravascular activation of neutrophils. A recent study shows that intravascular activation of neutrophils leads to the formation of Cys LT's in the cerebral microvasculature of isolated guinea pig brain. (DiGennaro et al., 2004, FASEB 18:842-844). Pretreatment of neutrophils with a leukotriene synthesis inhibitor prevented synthesis of Cys LT's, and prevented the development of cerebral edema. (DiGennaro et al.,) Hence, Cys-LT₂ receptor antagonists may be useful in treating the injuries associated with stroke, including cerebral edema.

### SUMMARY OF THE INVENTION

In one embodiment, this invention involves the use of compounds which are CysLT₂ receptor antagonists, or dual CysLT₁ and CysLT₂ receptor antagonists, to slow or halt atherogenesis or decrease myocardial infarction. Therefore, one object of the instant invention is to provide a method for treating atherosclerosis, which includes halting or slowing the progression of atherosclerotic disease once it has become clinically evident, comprising administering a therapeutically effective amount of a CysLT₂ receptor antagonist or dual CysLT₂ and CysLT₁ receptor antagonist to a patient in need of such treatment.

Another object is to provide methods for preventing or reducing the risk of developing atherosclerosis, comprising administering a prophylactically effective amount of a CysLT₂ receptor antagonist or dual CysLT₂ and CysLT₁ receptor antagonist to a patient who is at risk of developing atherosclerosis.

A further object is to provide the use of a CysLT₂ receptor antagonist or dual CysLT₂ and CysLT₁ receptor antagonist in combination with other anti-atherogenic drugs to prevent myocardial infarction and improve outcomes for patients.

In another embodiment, this invention is directed to the use of compounds which are CysLT₂ receptor antagonists, or dual CysLT₁ and CysLT₂ receptor antagonists, to treat pulmonary fibrosis. A further object is to provide the use of a CysLT₂ receptor antagonist or dual CysLT₂ and CysLT₁ receptor antagonist in combination with other drugs to treat pulmonary fibrosis.

In another embodiment, this invention is directed to the use of compounds which are CysLT₂ receptor antagonists, or dual CysLT₁ and CysLT₂ receptor antagonists, whether alone or in combination with other drugs, to treat stroke, including cerebral edema.

Additional objects will be evident from the following detailed description.

### DETAILED DESCRIPTION OF THE INVENTION

Atherosclerosis is characterized by the deposition of atheromatous plaques containing cholesterol and lipids on the innermost layer of the walls of large and medium-sized arteries. Atherosclerosis encompasses vascular diseases and conditions that are recognized and understood by physicians practicing in the relevant fields of medicine. Atherosclerotic cardiovascular disease, including restenosis following revascularization procedures, coronary heart disease (also known as coronary artery disease or ischemic heart disease), cerebrovascular disease including multi-infarct dementia, and peripheral vessel disease including erectile dysfunction, are all clinical manifestations of atherosclerosis and are therefore encompassed by the terms “atherosclerosis” and “atherosclerotic disease.”

One aspect of this invention involves a method for preventing or reducing the risk of developing atherosclerosis, comprising administering a prophylactically effective amount of a CysLT₂ receptor antagonist or dual CysLT₁ and CysLT₂ receptor antagonist to a patient in need of such treatment.

A CysLT₂ receptor antagonist or dual CysLT₂ and CysLT₁ receptor antagonist may be administered to prevent or reduce the risk of occurrence, or recurrence where the potential exists, of a coronary heart disease event, a cerebrovascular event, and/or intermittent claudication. Coronary heart disease events include CHD death, myocardial infarction (i.e., a heart attack), aortic aneurysms, and coronary revascularization procedures. Cerebrovascular events include ischemic or hemorrhagic stroke (also known as cerebrovascular accidents) and transient ischemic attacks. Intermittent claudication is a clinical manifestation of peripheral vessel disease. The term “atherosclerotic disease event” as used herein encompasses coronary heart disease events, cerebrovascular events, and intermittent claudication. It is intended that persons who have previously experienced one or more non-fatal atherosclerotic disease events are those for whom the potential for recurrence of such an event exists.

Accordingly, the instant invention also provides a method for preventing or reducing the risk of a first or subsequent occurrence of an atherosclerotic disease event comprising the administration of a prophylactically effective amount of a CysLT₂ receptor antagonist or dual CysLT₁ and CysLT₂ receptor antagonist to a patient at risk for such an event. The patient may already have atherosclerotic disease at the time of administration, or may be at risk for developing atherosclerotic disease.

The method of this invention particularly serves to prevent or slow new atherosclerotic lesion or plaque formation, and to prevent or slow progression of existing lesions or plaques, as well as to cause regression of existing lesions or plaques.

Accordingly, one aspect of this invention involves a method for halting or slowing the progression of atherosclerosis, including halting or slowing atherosclerotic plaque progression, comprising administering a therapeutically effective amount of a CysLT₂ receptor antagonist or dual CysLT₁ and CysLT₂ receptor antagonist to a patient in need of such treatment. This method also includes halting or slowing progression of atherosclerotic plaques existing at the time the instant treatment is begun (i.e., “existing atherosclerotic plaques”), as well as halting or slowing formation of new atherosclerotic plaques in patients with atherosclerosis.

Another aspect of this invention involves a method for regression of atherosclerosis, including regression of atherosclerotic plaques existing at the time the instant treatment is begun, comprising administering a therapeutically effective amount of a CysLT₂ receptor antagonist or dual CysLT₁ and CysLT₂ receptor antagonist to a patient in need of such treatment.

Another aspect of this invention involves a method for preventing or reducing the risk of atherosclerotic plaque rupture, comprising administering a prophylactically effective amount of a CysLT₂ receptor antagonist or dual CysLT₁ and CysLT₂ receptor antagonist to a patient in need of such treatment.

Another aspect of this invention involves a method for preventing or reducing the risk of aortic aneurysm formation, including atherosclerotic diet-induced aortic aneurysms, comprising administering a prophylactically effective amount of a CysLT₂ receptor antagonist or dual CysLT₁ and CysLT₂ receptor antagonist to a patient in need of such treatment.

As used herein, the term “aortic aneurysm” refers to a bulge in the wall of the aorta. Aortic aneurysms often occur in the abdomen (abdominal aortic aneurysm), but may also occur in the chest cavity (thoracic aortic aneurysm). Aortic aneurysms are typically caused by atherosclerosis.

In one embodiment, the invention is directed to treating, ameliorating or controlling stroke and the neurologic injuries caused by stroke.

As used herein, the term “stroke” refers to a clinical event involving impairment of cerebral circulation, that results in neurologic injury. Typically, stroke is manifest by the abrupt onset of a focal neurologic deficit. Stroke results from a rupture or obstruction (as by a thrombus or embolus) of an artery of the brain.

As used herein, the term “ischemic stroke” refers to stroke characterized by localized tissue anemia due to obstruction of the inflow of arterial blood. Ischemic stroke is usually caused by atherothrombosis or embolism of a major cerebral artery, but may also be caused by coagulation disorders or nonatheromatous vascular disease.

As used herein, the term “cerebral edema” refers to fluid collecting in brain tissue due to cellular swelling and the breakdown of the blood-brain barrier. Post-stroke cerebral edema may also involve the exuding of cerebrospinal fluid from ependymal lining, or the creation of an osmotic environment due to blood clots or tissue injury. The osmotic environment allows the movement of water into interstitial spaces. Post-stroke cerebral edema is often responsible for a worsening in the stroke patient's clinical status. Patients who have suffered stroke more than 24 hours previously often develop cerebral edema. Cerebral edema typically occurs at from one to five days after stroke.

One class of patients to which a compound of the invention may be administered is a patient at risk for stroke. As used herein, the term “patient at risk for stroke” means an individual who has had a previous stroke, or has a risk factor for stroke. Known risk factors for stroke include atherosclerosis, arterial hypertension, lipohyalinosis, hyperlipidemia, hypercholesterolemia, atrial fibrillation, smoking, inflammatory markers (including C-reactive protein), infection, homocysteine, sleep-disordered breathing, cerebral autosomal dominant arteriopathy with subcortial infarcts and leuko-encephalopathy (CADASIL), migraine, sickle-cell anemia, antiphospholipid antibody syndrome, arterial dissection, cocaine abuse and obesity.

Efforts at “controlling” stroke (including preventing stroke) can be divided into the primary prevention of stroke (treatment of patients who have not had any prior transient ischemic attacks of strokes, and have no neurological symptoms) and secondary prevention of stroke (treatment of patients who have had a prior transient ischemic attack or stroke). Primary prevention of stroke includes non-pharmacologic interventions, such as smoking cessation, healthy eating patterns, increased physical activity and weight management. Primary prevention also includes certain pharmacologic interventions, such as blood pressure control, treatment of atrial fibrillation, and management of diabetes, if appropriate. As part of the primary prevention of stroke, patients at high risk of coronary heart disease are often treated with aspirin. As part of primary prevention, patients having high amounts of low density lipoprotein (LDL) are often subject to blood lipid management, to reduce LDL levels to acceptable levels, e.g. below 160 mg/dl.

The secondary prevention of stroke often involves the same pharmacologic and non-pharmacologic interventions used for primary prevention, including blood pressure control, treatment of atrial fibrillation, management of diabetes, treatment with aspirin, and blood lipid management. Additional common secondary prevention interventions include the use of antiplatelet agents (such as clopidrogel), anticoagulants (such as warfarin), and anti-hypertension agents (such as beta andrenergic antagonists).

A second class of patients to which a compound of the invention may be administered are acute stroke patients, i.e., patients who have suffered ischemic stroke within the last 7 days. One preferred class of acute stroke patients are those who have suffered stroke within the last 3 days. A more preferred class of acute stroke patients are those who have suffered stroke within the last 48 hours, even more preferably within the last 24 hours. As common in the art of treating stroke, patients may be classified according to the period of time when stroke occurred. So, for example, one class of acute stroke patients are those who have suffered stroke within the last 18 hours. Another class of acute stroke patients are those who have suffered stroke within the last 12 hours. Another class of acute stroke patients are those who have suffered stroke within the last 8 hours. Another class of acute stroke patients are those who have suffered stroke within the last 6 hours. Another class of acute stroke patients are those who have suffered stroke within the last 4 hours. Another class of acute stroke patients are those who have suffered stroke within the last 3 hours.

The compounds included within the scope of this invention are CysLT₂ receptor antagonists, including selective CysLT₂ antagonists and dual CysLT₂ and CysLT₁ receptor antagonists. In general, CysLT₂ antagonists can be identified as those compounds which when assayed in the assay described in Example 10 below have an IC₅₀ of less than or equal to 500 nM. Preferred CysLT₂ antagonists have an IC₅₀ of less than or equal to 100 nM, more preferably less than or equal to 50 nM, most probably less than or equal to 10 nM.

In one embodiment, the CysLT₂ antagonist is a dual CysLT₂ and CysLT₁ antagonist. Suitable dual CysTL₂ and CysTL₁ receptor antagonists have an IC₅₀ of less than or equal to 500 nM for the CysLT₂ receptor, and less than or equal to 500 nM for the CysLT₁ receptor. A preferred dual antagonist has an IC₅₀ of less than or equal to 500 nM for the CysLT₂ receptor, and an IC₅₀ of less than or equal to 100 nM, more preferably 50 nM, most preferably 10 nM, for the CysLT₁ receptor.

Another preferred dual antagonist has an IC₅₀ of less than or equal to 100 nM for the CysLT₂ receptor, and an IC₅₀ of less than or equal to 100 nM, more preferably 50 nM, most preferably 10 nM, for the CysLT₁ receptor.

Another preferred dual antagonist has an IC₅₀ of less than or equal to 50 nM for the CysLT₂ receptor, and an IC₅₀ of less than or equal to 100 nM, more preferably 50 nM, most preferably 10 nM, for the CysLT₁ receptor.

Another preferred dual antagonist has an IC₅₀ of less than or equal to 10 nM for the CysLT₂ receptor, and an IC₅₀ of less than or equal to 100 nM, more preferably 50 nM, most preferably 10 nM, for the CysLT₁ receptor.

In another embodiment, the dual CysLT₂ and CysLT₁ receptor antagonist has an IC₅₀ of less than or equal to 500 nM for the CysLT₁ receptor, and less than or equal to 500 nM for the CysLT₂ receptor. A preferred dual antagonist has an IC₅₀ of less than or equal to 500 nM for the CysLT₁ receptor, and an IC₅₀ of less than or equal to 100 nM, more preferably 50 nM, most preferably 10 nM, for the CysLT₂ receptor.

Another preferred dual antagonist has an IC₅₀ of less than or equal to 100 nM for the CysLT₁ receptor, and an IC₅₀ of less than or equal to 100 nM, more preferably 50 nM, most preferably 10 nM, for the CysLT₂ receptor.

Another preferred dual antagonist has an IC₅₀ of less than or equal to 50 nM for the CysLT₁ receptor, and an IC₅₀ of less than or equal to 100 nM, more preferably 50 nM, most preferably 10 nM, for the CysLT₂ receptor.

Another preferred dual antagonist has an IC₅₀ of less than or equal to 10 nM for the CysLT₁ receptor, and an IC₅₀ of less than or equal to 100 nM, more preferably 50 nM, most preferably 10 nM, for the CysLT₂ receptor.

In general, selective CysLT₂ antagonists are selective to the CysLT₂ receptor in comparison to the CysLT₁ receptor. In one embodiment, the selective CysLT₂ antagonist possesses a selectivity for the CysLT₂ receptor relative to the CysLT₁ receptor of at least 5 fold intrinsic binding affinity.

In another embodiment, the selective CysLT₂ antagonist possesses a selectivity for the CysLT₂ receptor relative to the CysLT₁ receptor of at least 10 fold intrinsic binding affinity.

In another embodiment, the selective CysLT₂ antagonist possesses a selectivity for the CysLT₂ receptor relative to the CysLT₁ receptor of at least 50 fold intrinsic binding affinity.

In another embodiment, the selective CysLT₂ antagonist possesses a selectivity for the CysLT₂ receptor relative to the CysLT₁ receptor of at least 100 fold intrinsic binding affinity.

In another embodiment, the selective CysLT₂ antagonist possesses a selectivity for the CysLT₂ receptor relative to the CysLT₁ receptor of at least 200 fold intrinsic binding affinity.

In another embodiment, the selective CysLT₂ antagonist possesses a selectivity for the CysLT₂ receptor relative to the CysLT₁ receptor of at least 500 fold intrinsic binding affinity.

Examples of CysLT₂ antagonists useful in the invention are compounds (I) ((3-((carboxyacetal)amino)phenyl)thio)-4-nonyl-oxobenzenehexanoic acid) and (II) ((2S, 3S, 2′S,3′S)-3,3′-[({3-[(E)-2-(7-chloroquinolin-2-yl)vinyl]phenyl}methylene)bis(thio)]bis(2-methylbutanoic acid), shown below:

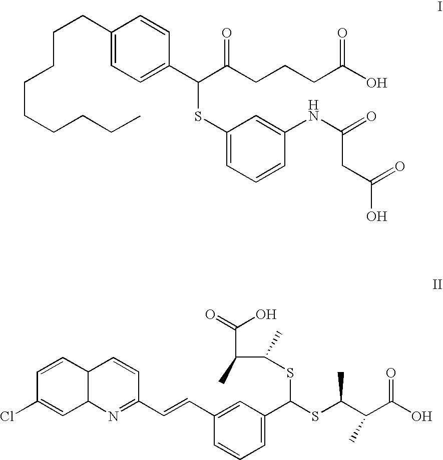

As used herein, the term “binding affinity” is a measure of the physicochemical interaction between a radiolabelled ligand and its specific receptor in vitro. One measure of binding affinity is the inhibitory concentration or IC₅₀ value, which is the concentration of unlabeled radioligand (or ligand of interest, for example a CysLT2 receptor antagonist of the type contemplated for use in this invention) which is required to inhibit 50% of the specific binding of the radiolabelled radioligand. The IC₅₀ value can be determined by various competitive binding assays known to those skilled in the art.

As used herein, the term “patient” includes mammals, especially humans, who use the instant active agents for the prevention or treatment of a medical condition. Administering of the drug to the patient includes both self-administration and administration to the patient by another person. For example, in the treatment of atherosclerosis, the patient may be in need of treatment for an existing atherosclerosis-related disease or medical condition, or may desire prophylactic treatment to prevent or reduce the risk of onset of atherosclerosis, or atherosclerosis medical condition or atherosclerosis disease event.

As used herein, the term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician.

An effective amount of a CysLT₂ receptor antagonist or dual CysLT₁ and CysLT₂ receptor antagonist in the methods of this invention is in the range of about 0.001 mg/kg to about 20 mg/kg of body weight per day, preferably 0.01 mg to about 10 mg per kg, and most preferably 0.1 to 1 mg per kg, in single or divided doses. A single daily dose is preferred but not necessary. On the other hand, it may be necessary to use dosages outside these limits in some cases. As examples, the daily dosage amount may be selected from, but not limited to, 25 mg, 50 mg, 75 mg, 100 mg, 125 mg, 150 mg, 200 mg and 250 mg. It will be understood, however, that the specific dose level for any particular patient will depend upon a variety of factors including the disease or disorder to be treated, age, body weight, general health, sex, diet, time of administration, route of administration, rate of excretion, drug combination and the severity of the patient's condition. A consideration of these factors is well within the purview of the ordinarily skilled clinician for the purpose of determining the therapeutically effective or prophylactically effective dosage amount needed to prevent, counter, or arrest the progress of the condition. It is expected that the CysLT₂ receptor antagonist or dual CysLT₁ and CysLT₂ receptor antagonist will be administered chronically on a daily basis for a length of time appropriate to treat or prevent the medical condition relevant to the patient, including a course of therapy lasting months, years or the life of the patient.

In the methods of treatment of this invention, the CysLT₂ receptor antagonist or dual CysLT₁ and CysLT₂ receptor antagonist may be administered via any suitable route of administration such as orally, parenterally, or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques. Oral formulations are preferred.

For oral use, the pharmaceutical compositions of this invention containing the active ingredient may be in forms such as tablets, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc.

Oral immediate-release and time-controlled release dosage forms may be employed, as well as enterically coated oral dosage forms. Tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. One example of a time-controlled release device is described in U.S. Pat. No. 5,366,738. They may also be coated by the technique described in U.S. Pat. Nos. 4,256,108; 4,166,452; and 4,265,874, to form osmotic therapeutic tablets for controlled release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water or miscible solvents such as propylene glycol, PEGs and ethanol, or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin, or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. Cosolvents such as ethanol, propylene glycol or polyethylene glycols may also be used. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds useful in the method of treatment of the invention may also be administered in the form of a suppository for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

In the treatment of atherosclerosis, one or more additional active agents, for example but not limited to anti-atherosclerotic agents, may be used in combination with the CysLT₂ receptor antagonists or a dual CysLT₁ and CysLT₂ receptor antagonist of this invention in a single dosage formulation, or may be administered to the patient in a separate dosage formulation, which allows for concurrent or sequential administration of the active agents. The additional active agent or agents can be lipid altering compounds such as HMG-CoA reductase inhibitors, or agents having other pharmaceutical activities, or agents that have both lipid-altering effects and other pharmaceutical activities. Examples of HMG-CoA reductase inhibitors useful for this purpose include statins in their lactonized or dihydroxy open acid forms and pharmaceutically acceptable salts and esters thereof, including but not limited to lovastatin (see U.S. Pat. No. 4,342,767); simvastatin (see U.S. Pat. No. 4,444,784); dihydroxy open-acid simvastatin, particularly the ammonium or calcium salts thereof; pravastatin, particularly the sodium salt thereof (see U.S. Pat. No. 4,346,227); fluvastatin, particularly the sodium salt thereof (see U.S. Pat. No. 5,354,772); atorvastatin, particularly the calcium salt thereof (see U.S. Pat. No. 5,273,995); nisvastatin, also referred to as NK-104 (see PCT international publication number WO 97/23200); and rosuvastatin (see U.S. Pat. No. 5,260,440).

Additional active agents which may be used in combination with a CysLT₂ receptor antagonist or dual CysLT₁ and CysLT₂ receptor antagonist for treatment of atherosclerosis include, but are not limited to, HMG-CoA synthase inhibitors; cholesterol absorption inhibitors such as ezetimibe which is 1-(4-fluorophenyl)-3(R)-[3(S)-(4-fluorophenyl)-3-hydroxypropyl)]-4(S)-(4-hydroxyphenyl)-2-azetidinone, described in U.S. Pat. No. Re. 37,721 and U.S. Pat. No. 5,846,966; cholesterol ester transfer protein (CETP) inhibitors, for example JTT-705 and CP529,414; squalene epoxidase inhibitors; squalene synthetase inhibitors (also known as squalene synthase inhibitors); acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; probucol; niacin; bile acid sequestrants; LDL (low density lipoprotein) receptor inducers; platelet aggregation inhibitors, for example glycoprotein IIb/IIIa fibrinogen receptor antagonists and aspirin; human peroxisome proliferator activated receptor gamma (PPARγ) agonists, including the compounds commonly referred to as glitazones, for example troglitazone, pioglitazone and rosiglitazone and including those compounds included within the structural class known as thiazolidinediones as well as those PPARγ agonists outside the thiazolidinedione structural class; PPARα agonists such as clofibrate, fenofibrate including micronized fenofibrate, and gemfibrozil; PPAR dual α/γ agonists such as 5-[(2,4-dioxo-5-thiazolidinyl)methyl]-2-methoxy-N-[[4-(trifluoromethyl)phenyl]methyl]-benzamide, known as KRP-297; vitamin B6 (also known as pyridoxine) and the pharmaceutically acceptable salts thereof such as the HCl salt; vitamin B12 (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; calcium channel blockers such as nifedipine and diltiazam; endothelian antagonists; agents that enhance ABC1 gene expression; FXR and LXR ligands including both inhibitors and agonists; bisphosphonate compounds such as alendronate sodium; and cyclooxygenase-2 inhibitors such as rofecoxib and celecoxib.

In the treatment of stroke, one or more additional active agents may be used in combination with the CysLT₂ receptor antagonists or a dual CysLT₁ and CysLT₂ receptor antagonist of this invention in a single dosage formulation, or may be administered to the patient in a separate dosage formulation, which allows for concurrent or sequential administration of the active agents.

Additional active agents which may be used in combination with a CysLT2 receptor antagonist or dual CysLT1 and CysLT2 receptor antagonists in the treatment of stroke include, but are not limited to, COX-2 inhibitors; nitric oxide synthase inhibitors, such as N-(3-(aminomethyl)benzyl)acetamidine (1400W94); Rho kinase inhibitors, such as fasudil; angiotension II type-1 receptor antagonists, including candesartan (marketed as ATACAND™), losartan (marketed as COZAAR™), irbesartan (marketed as AVAPRO™), eprosartan (marketed as TEVETEN™), telmisartan (marketed as MICARDIS™) and valsartan (marketed as DIOVAN™); glycogen synthase kinase 3 inhibitors; sodium or calcium channel blockers, including crobenetine; p38 MAP kinase inhibitors, including SKB 239063; thromboxane AX-synthetase inhibitors, including isbogrel, ozagrel, ridogrel and dazoxiben; statins (HMG CoA reductase inhibitors), including lovastatin, simvastatin, dihydroxy open-acid simvastatin, pravastatin, fluvastatin, atorvastatin, nisvastatin, and rosuvastatin; neuroprotectants, including free radical scavengers, calcium channel blockers, excitatory amino acid antagonists, growth factors, antioxidants, such as edaravone, vitamin C, TROLOX™, citicoline and minicycline, and reactive astrocyte inhibitors, such as (2R)-2-propyloctanoic acid; beta andrenergic blockers, such as propranolol, nadolol, timolol, pindolol, labetalol, metoprolol, atenolol, esmolol and acebutolol; NMDA receptor antagonists, including memantine; NR2B antagonists, such as traxoprodil; 5-HT1A agonists; receptor platelet fibrinogen receptor antagonists, including tirofiban and lamifiban; thrombin inhibitors; antithrombotics, such as argatroban; antihypertensive agents, such as enalapril; vasodilators, such as cyclandelate; nociceptin antagonists; DPIV antagonists; GABA 5 inverse agonists; and selective androgen receptor modulators.

In the treatment of pulmonary fibrosis, one or more additional active agents may be used in combination with the CysLT₂ receptor antagonists or a dual CysLT₁ and CysLT₂ receptor antagonist of this invention in a single dosage formulation, or may be administered to the patient in a separate dosage formulation, which allows for concurrent or sequential administration of the active agents. Additional active agents which may be used in combination with a CysLT2 receptor antagonist or dual CysLT1 and CysLT2 receptor antagonists in the treatment of pulmonary fibrosis include, but are not limited to, anti-inflammatory agents, such as corticosteroids, azathioprine or cyclophosphamide.

In one embodiment, the invention is further directed to a method for the manufacture of a medicament or a composition for treating or preventing atherosclerosis, or an atherosclerosis medical condition or atherosclerosis related medical event, in humans and animals, comprising combining a CysLT₂ receptor antagonist with a pharmaceutical carrier or diluent. In one embodiment, the CysLT₂ receptor antagonist is a selective CysTL₂ receptor antagonist. In another embodiment, the CysLT₂ receptor antagonist is a dual CysLT₁ and CysLT₂ receptor antagonist.

In another embodiment, the invention is directed to a method for the manufacture of a medicament or a composition for treating pulmonary fibrosis in humans and animals, comprising combining a CysLT₂ receptor antagonist with a pharmaceutical carrier or diluent. In one embodiment, the CysLT₂ receptor antagonist is a selective CysTL₂ receptor antagonist. In another embodiment, the CysLT₂ receptor antagonist is a dual CysLT₁ and CysLT₂ receptor antagonist.

In another embodiment, the invention is directed to a method for the manufacture of a medicament or a composition for treating stroke (including cerebral edema) in humans and animals, comprising combining a CysLT₂ receptor antagonist with a pharmaceutical carrier or diluent. In one embodiment, the CysLT₂ receptor antagonist is a selective CysTL₂ receptor antagonist. In another embodiment, the CysLT₂ receptor antagonist is a dual CysLT₁ and CysLT₂ receptor antagonist.

A synthesis of compound (I) is shown in the Scheme below: 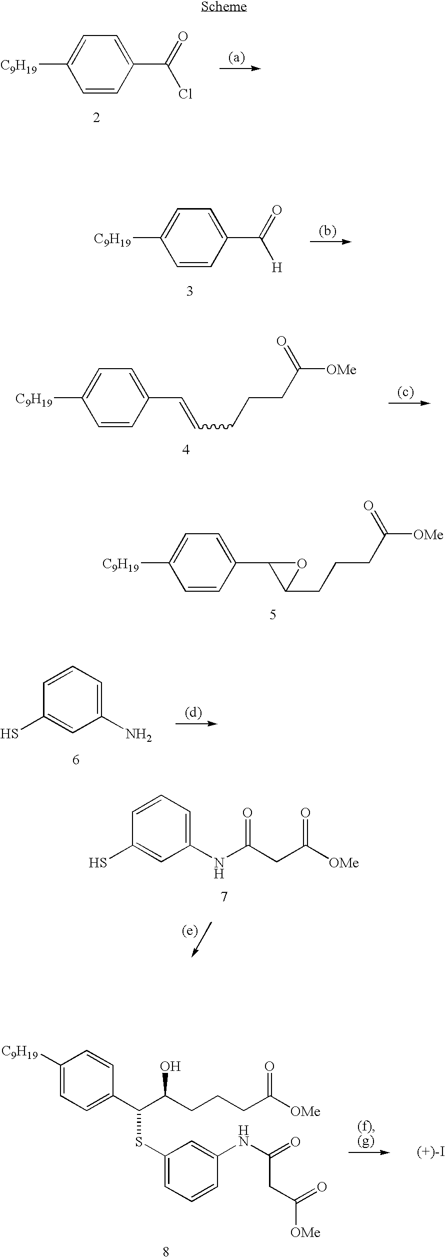
Reagents: (a) Ph₃P, (Ph₃P)₂CuBH₄, acetone; (b) Ph₃P⁺(CH₂)₄CO₂H Br⁻, KOt-Bu, THF; then HCl, MeOH; (c) m-CPBA, CH₂Cl_{2;} (d) diethyl malonate, 170° C.; (e) 5, Et₃N, MeOH; (f) pyridinium chlorochromate, NaOAc, CH₂Cl₂; (g) NaOH, MeOH then HCl.

In the Scheme above, the diacid 1 is obtained by reduction of the commercially available acid chloride 2 (Alfa Aesar) to the aldehyde 3 using bis(triphenylphosphine)copper (I) borohydride in methanol at ambient temperature. A Wittig reaction between aldehyde 3 and the ylid derived from 5-(triphenylphosphonium)pentanoic acid bromide followed by esterification with methanol then yielded the ester 4. The ratio of cis- to trans-double bond isomers was approximately 1:1 and this mixture was used in the epoxidation step using m-CPBA as the oxidant. Preparative liquid chromatography was then used to separate the resulting cis- and trans-epoxides. The trans-epoxide 5 was coupled with thiol 7 (obtained by condensation of 3-aminothiophenol 6 with diethyl malonate) to give the alcohol 8. Compound 8 was oxidized to the corresponding ketone with pyridinium chlorochromate and the product hydrolysed with base to afford 1,((3-((carboxyacetal)amino)phenyl)thio)-4-nonyl-oxobenzenehexanoic acid), as a racemate. The enantiomeric separation of 1 was achieved using a Chiralcel OD column eluting with 0.1% TFA in hexane/methanol/1-propanol in the ratio 90/5/5.

A synthesis of compound (II), (2S, 3S, 2′S, 3′S)-3,3′-[({3-(E)-2-(2-chloroquinolin-2-yl)vinyl]phenyl}methylene)bis(thio)]bis(2-methylbutanoic acid), is described in Examples 5-9 below.

The starting materials and reagents for the processes described herein are either commercially available or are known in the literature or may be prepared following literature methods described for analogous compounds. The skills required in carrying out the reaction and purification of the resulting reaction products are known to those skilled in the art. Purification procedures include crystallization, distillation, normal phase or reverse phase chromatography.

### EXAMPLE 1

### Ethyl 3-[(3-mercaptophenyl)amino]-3-oxopropanoate

A solution of 3-aminothiophenol (5.0 g, 40 mmol) in diethyl malonate (40 mL) under N₂ atmosphere was heated for 2 hours at 165° C. The total mixture was purified by silica gel chromatography eluting first with CHCl₃, then 1% MeOH in CHCl₃ followed by 4% MeOH in CHCl₃ giving the title compound (m.p. 52° C.-54° C.).

Analysis calculated for C₁₁H₁₃NO₃S: C, 55.21; H, 5.47; N, 5.85; S, 13.39. Found: C, 54.64; H, 5.41; N, 5.80; S, 13.02.

### EXAMPLE 2

### Methyl (5S, 6R)-5-hydroxy-6-({3-[(3-methoxy-3-oxopropanoyl)amino]phenyl}thio)-6-(4-nonylphenyl)hexanoate

A solution of methyl 4-[(2R)-3-(4-nonylphenyl)oxiran-2-yl]butanoate (1.5 g, 4.3 mmol) (which is disclosed in in EP 0123543), ethyl 3-[(3-mercaptophenyl)amino]-3-oxopropanoate (1.0 g, 4.3 mmol) and triethylamine (2.1 mL) in methanol (30 mL) was stirred for 18 hours in a stoppered flask at room temperature. The solution was concentrated and the residue purified by silica gel chromatography eluting with 50% ethyl acetate in hexanes to give the title compound as an oil.

Analysis calculated for C₃₂H₄₅NO₆S: C, 67.22; H, 7.93; N, 2.44; S, 5.60. Found: C, 67.06; H, 8.06; N, 2.36; S, 5.83.

### EXAMPLE 3

### Methyl 6-({3-[(3-methoxy-3-oxopropanoyl) amino]phenyl}thio)-6-(4-nonylphenyl)-5-oxohexanoate

To a solution of methyl (5S, 6R)-5-hydroxy-6-({3-[(3-methoxy-3-oxopropanoyl) amino]phenyl}thio)-6-(4-nonylphenyl)hexanoate (2.0 g, 3.5 mmol), in dichloromethane (100 mL) was added anhydrous sodium acetate (570 mg, 7.0 mmol) and pyridinium chlorochromate (3.2 g, 14.0 mmol). The mixture was stirred for 2.5 hours at room temperature diluted with diethyl ether (500 mL), filtered through Celite and the filtrate concentrated. Purification by silica gel chromatography eluting with 50% ethyl acetate in hexanes gave the title compound (m.p. 88° C.-91° C.).

Analysis calculated for C₃₂H₄₃NO₆S: C, 67.45; H, 7.60; N, 2.45; S, 5.62. Found: C, 67.12; H, 7.79; N, 2.44; S, 5.78.

### EXAMPLE 4

### 6-({3-[(carboxyacetyl)amino]phenyl}thio)-6-(4-nonylphenyl)-5-oxohexanoic acid

A solution of methyl 6-({3-[(3-methoxy-3-oxopropanoyl)amino]phenyl}thio)-6-(4-nonylphenyl)-5-oxohexanoate (815 mg, 1.4 mmol) and 1N NaOH (4.5 mL, 4.5 mmol) in methanol (25 mL) was stirred at room temperature for 18 hours. The mixture was concentrated to remove the methanol and then acidified with 1N HCl (5.0 mL). The mixture was extracted with diethyl ether (50 mL) dried (Na₂SO₄), filtered, concentrated and the residue purified by silica gel chromatography eluting with 4:8:1 methanol: chloroform: ammonium hydroxide. The purified title compound as the ammonium salt was acidified with 1N HCl, extracted with diethyl ether, dried (Na₂SO₄), filtered and concentrated to give the title compound (m.p. 88° C.-91° C.).

Analysis calculated for C₃₀H₃₉NSO₆: C, 66.51; H, 7.25; N, 2.58; S, 5.91. Found: C, 66.64; H, 7.53; N, 2.67; S, 6.04.

### EXAMPLE 5

### Methyl (2R,3R)-3-hydroxy-2-methylbutanoate

Methyl lithium (300 mL, 1.4M/ether, 418 mmol) was added to diisopropylamine (62.9 mL, 449 mmol) at −20° C. and the slurry was stirred for 15 min before diluting with THF (140 mL) and cooling to −60° C. Methyl 3R-hydroxybutanoate (24.3 g, 204 mmol) was slowly added as a THF (10 mL) solution and the resulting solution was stirred 45 min allowing the temperature to rise to −35° C. After cooling again to −70° C., methyl iodide (75.7 mL, 816 mmol) was added and the reaction mixture was stirred overnight with slow warming to +10° C. Ice cold 1N HCl (300 mL) was added and the aqueous phase was extracted with ether (3×300 mL). The combined organic layers were washed with 10% Na₂S₂O₃, brine, dried over MgSO₄ and concentrated in vacuo. Distillation under reduced pressure gave the title compound as an oil. [α]_{D}=−28.70° (c=2.0, acetone).

¹H NMR (250 MHz, acetone-d6) δ3.9 (1H, m), 3.75 (1H, br d), 3.6 (3H, s), 2.45 (1H, m), 1.11 (3H, d), 1.07 (3H, d).

### EXAMPLE 6

### Methyl (2S,3S)-3-acetylthio-2-methylbutanoate

Diethyl azodicarboxylate (27.2 mL, 172 mmol) was added to THF solution (350 mL) of triphenyl phosphine (45.2 g, 172 mmol) at −10° C. The mixture was stirred mechanically for 1 h at that temperature. The resulting thick paste was cooled to −30° C. and methyl (2R,3R)-3-hydroxy-2-methylbutanoate was slowly added as a THF solution (40 mL) followed by thioacetic acid (12.3 mL, 172 mmol). The reaction mixture was stirred overnight at 0° C. Solids were filtered off, rinsed with ether and the filtrate was concentrated in vacuo. Purification by silica gel flash chromatography eluting with 0 to 1.5% Et₂O/CH₂Cl₂) gave the title compound as an oil. [α]_{D}=−12.50 (c=2.0, acetone).

¹H NMR (250 MHz, acetone-d6) δ3.8 (1H, m), 3.62 (3H, s), 2.68 (1H, m), 2.3 (3H, s) 1.3 (3H, d), 1.15 (3H, s).

### EXAMPLE 7

### (2S,3S)-3-acetylthio-2-methylbutanoic acid

Lithium iodide (15.4 g, 115 mmol) was dissolved in DMF (15 mL) at 125° C. for 1 h. Methyl (2S,3S)-3-(acetylthio)-2-methylbutanoate was then added as a DMF solution (8 mL) and the reaction mixture was stirred vigorously at 125 ° C. for 16 h. Water (100 mL) was added and the mixture extracted with ethyl acetate (2×150 mL). The combined organic layers were washed with 10% aq. Na₂S₂O₃, brine, dried over MgSO₄ and concentrated invacuo. High vacuum distillation to remove the DMF left the title compound as a slightly brownish oil.

¹H NMR (250 MHz, acetone-d6) δ3.32 (1H, m), 2.62 (1H, m), 1.8 (3H, s), 1.33 (3H, d), 1,19 (3H, d).

### EXAMPLE 8

### (2S,3S)-3-mercapto-2-methylbutanoic acid

(2S,3S)-3-Acetylthio-2-methylbutanoic acid (5.9 g, 33.5 mmol) was dissolved in methanol (40 mL) and cooled to −5° C. Nitrogen was bubbled through the solution for 15 min. Aqueous sodium hydroxide (10 N, 10 mL, 100 mmol) was added and the solution was stirred 45 min at −5° C. The reaction mixture was cooled to −40° C. and HCl conc. (10 mL, ˜120 mmol) was slowly added. The mixture was extracted with ethyl acetate (4×150 mL), the combined organic layers were washed with brine, dried over MgSO₄ and concentrated in vacuo. Distillation under high vacuum afforded the title compound as an oil. [α]_{D}=+19.3° (c=2.0, acetone).

¹H NMR (250 MHz, acetone-d6) δ3.3 (1H, m), 2.53 (1H, m), 1.9 (1H, d), 1.35 (3H, d), 1.2 (3H, d).

### EXAMPLE 9

### (2S, 3S, 2′S,3′S)-3,3′-[({3-[(E)-2-(7-chloroquinolin-2-yl)vinyl]phenyl}methylene)bis(thio)]bis(2-methylbutanoic acid)

To a solution of 3-[(E)-2-(7-chloroquinolin-2-yl)vinyl]benzaldehyde (875 mg, 3 mmol) (which is disclosed in U.S. Pat. No. 4,851,409) in trifluoroacetic acid (4 mL) was slowly added dropwise a solution of (2S,3S)-3-mercapto-2-methylbutanoic acid (800 mg, 6 mmol) in trifluoroacetic acid (1 mL). The reaction mixture was stirred at room temperature for 15 min. and then poured into water (75 mL) and extracted with ethyl acetate (3×50 mL). The organic layer was evaporated and the residue purified by chromatography on silicic acid eluting with 25% to 35% acetone in toluene to give the title compound as a white foam.

¹H NMR (250 MHz, acetone-d6) δ8.45 (1H, d), 8.1-7.9 (5H, m), 7.7-7.52 (4H, m), 7.43 (1H, t), 5.36 (1H, s), 3.2 (2H, m), 2.65 (2H, m), 1.4 (3H, d), 1.32 (9H, m).

### EXAMPLE 10

### CysLT₂ Receptor Screen

Compounds may be screened for effects on LTD₄ induced calcium mobilization in cells transfected with CysLT₂ receptor, according to the following calcium mobilization assay.

Human embryonic kidney (HEK293T) cells transiently transfected with the CysLT₂ receptor using LipofectAMINE™2000 reagent (Life Technologies) or Chinese hamster ovary (CHO-NFAT) cells stably transfected with the CysLT₂ type receptor are plated into Poly-D-Lysine treated black-wall microplates (Biocoat™) at 5×104 cells per well. Cells are maintained for approximately 24 hours at 37° C. and 5% CO₂. After 24 hours, cells are loaded with Fluo4 calcium indicator dye (Molecular Probes) in the presence of 2.5 mM probenecid for one hour. After washing, the cells are treated with agonist, (10 nM LTD_{4,} BioMol), and maximum fluorescence measured in a Molecular Devices Fluorometric Imaging Plate Reader (FLIPR). Compounds screened for antagonism are added five minutes prior to addition of agonist. IC₅₀ values are determined for compounds exhibiting greater than 50% antagonism at 5 μM.

Compounds of interest from the CysLT₂ receptor screen may also be assayed in a CysLT₁ receptor counter screen. Compounds of interest may be assayed using the same protocol described above in cells transfected with the CysLT₁ receptor.

### EXAMPLE 11

Agonist and Standard Antagonist Functional Characterization on Human CysLT₁ and CysLT₂ Receptors

Compound (I) demonstrated an antagonist dose-response curve of blocking LTD₄-induced calcium flux in CysLT₁ receptor transfected HEK293T cells, with an IC₅₀ of less than 10 μM. Similar antagonist dose-response curves for CysLT₂ receptor-transfected cells also demonstrated an IC₅₀ of less than 10 μM.

The following abbreviations are used throughout the text:
t-Bu: tertiary butyl
Me: methyl
Et: ethyl
Ph: phenyl
Ac: acetyl
CPBA: chloroperoxybenzoic acid
THf: tetrahydrofuran
TFA: trifluoroacetic acid
DMF: N,N-dimethylformamide

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation employed, and such expected variations or differences in the results are contemplated in accordance with the objects and practices of the present invention. It is intended, therefore, that the invention be defined by the scope of the claims which follow and that such claims be interpreted as broadly as is reasonable.

1. A method of treating atherosclerosis, comprising administering a therapeutically effective amount of a cysteinyl leukotriene 2 receptor antagonist to a patient in need of such treatment.

2. The method of claim 1 wherein the cysteinyl leukotriene 2 receptor antagonist is a dual cysteinyl leukotriene 1 receptor antagonist and cysteinyl leukotriene 2 receptor antagonist.

3. The method of claim 1 wherein the cysteinyl leukotriene 2 receptor antagonist is a selective cysteinyl leukotriene 2 receptor antagonist.

4. The method of claim 1, wherein the cysteinyl leukotriene 2 receptor antagonist is selected from the group consisting of:
3-((carboxyacetal)amino)phenyl)thio)4-nonyl-oxobenzenehexanoic acid, and (2S, 3S, 2′S,3′S)-3,3′-[({3-[(E)-2-(7-chloroquinolin-2-yl)vinyl]phenyl}methylene)bis(thio)]bis(2-methylbutanoic acid.

5. A method for preventing or reducing the risk of atherosclerotic plaque rupture comprising administering a prophylactically effective amount of a cysteinyl leukotriene 2 receptor antagonist to a patient in need of such treatment.

6. The method of claim 5 wherein the cysteinyl leukotriene 2 receptor antagonist is a dual cysteinyl leukotriene 1 receptor antagonist and cysteinyl leukotriene 2 receptor antagonist.

7. The method of claim 5 wherein the cysteinyl leukotriene 2 receptor antagonist is a selective cysteinyl leukotriene 2 receptor antagonist.

8. The method of claim 5, wherein the cysteinyl leukotriene 2 receptor antagonist is selected from the group consisting of:
3-((carboxyacetal)amino)phenyl)thio)-4-nonyl-oxobenzenehexanoic acid, and
(2S, 3S, 2′S,3′S)-3,3′-[({3-[(E)-2-(7-chloroquinolin-2-yl)vinyl]phenyl}methylene)bis(thio)]bis(2-methylbutanoic acid.

9. A method of treating an aortic aneurysm, comprising administering a therapeutically effective amount of a cysteinyl leukotriene 2 receptor antagonist to a patient in need of such treatment.

10. The method of claim 9 wherein the cysteinyl leukotriene 2 receptor antagonist is a dual cysteinyl leukotriene 1 receptor antagonist and cysteinyl leukotriene 2 receptor antagonist.

11. The method of claim 9 wherein the cysteinyl leukotriene 2 receptor antagonist is a selective cysteinyl leukotriene 2 receptor antagonist.

12. The method of claim 9, wherein the cysteinyl leukotriene 2 receptor antagonist is selected from the group consisting of:
3-((carboxyacetal)amino)phenyl)thio)-4-nonyl-oxobenzenehexanoic acid, and
(2S, 3S, 2′S,3′S)-3,3′-[({3-[(E)-2-(7-chloroquinolin-2-yl)vinyl]phenyl}methylene)bis(thio)]bis(2-methylbutanoic acid.

13. A method of treating pulmonary fibrosis, comprising administering a therapeutically effective amount of a cysteinyl leukotriene 2 receptor antagonist to a patient in need of such treatment.

14. The method of claim 13 wherein the cysteinyl leukotriene 2 receptor antagonist is a dual cysteinyl leukotriene 1 receptor antagonist and cysteinyl leukotriene 2 receptor antagonist.

15. The method of claim 13 wherein the cysteinyl leukotriene 2 receptor antagonist is a selective cysteinyl leukotriene 2 receptor antagonist.

16. The method of claim 13, wherein the cysteinyl leukotriene 2 receptor antagonist is selected from the group consisting of:
3-((carboxyacetal)amino)phenyl)thio)-4-nonyl-oxobenzenehexanoic acid, and
(2S, 3S, 2′S,3′S)-3,3′-[({3-[(E)-2-(7-chloroquinolin-2-yl)vinyl]phenyl}methylene)bis(thio)]bis(2-methylbutanoic acid.

17. A method of treating cerebral edema, comprising administering a therapeutically effective amount of a cysteinyl leukotriene 2 receptor antagonist to a patient in need of such treatment.

18. The method of claim 17 wherein the cysteinyl leukotriene 2 receptor antagonist is a dual cysteinyl leukotriene 1 receptor antagonist and cysteinyl leukotriene 2 receptor antagonist.

19. The method of claim 17 wherein the cysteinyl leukotriene 2 receptor antagonist is a selective cysteinyl leukotriene 2 receptor antagonist.

20. The method of claim 17, wherein the cysteinyl leukotriene 2 receptor antagonist is selected from the group consisting of:
3-((carboxyacetal)amino)phenyl)thio)-4-nonyl-oxobenzenehexanoic acid, and
(2S, 3S, 2′S,3′S)-3,3′-[({3-[(E)-2-(7-chloroquinolin-2-yl)vinyl]phenyl}methylene)bis(thio)]bis(2-methylbutanoic acid.
